(19) United States
(12) Patent Application Publication
(10) Pub. No.: US 2023/0062278 A1
(43) Pub. Date: Mar. 2, 2023

(54) COMPOUNDS FOR THE TREATMENT OF MYELOFIBROSIS

(71) Applicant: ACTUATE THERAPEUTICS, INC., Fort Worth, TX (US)

(72) Inventors: Francis J. GILES, Chicago, IL (US); Andrew MAZAR, Lake Forest, IL (US)

(21) Appl. No.: 17/789,321

(22) PCT Filed: Dec. 23, 2020
(86) PCT No.: PCT/US2020/066762

§ 371 Date: Jun. 27, 2022

Related U.S. Application Data
(60) Provisional application No. 62/953,654, filed on Dec. 26, 2019.

(51) Int. Cl. A61K 31/407; A61K 31/519

(52) U.S. Cl. CPC A61K 31/407; A61K 31/519

(57) ABSTRACT

The present invention provides methods of treating myelofibrosis in a patient by administering to the patient a therapeutically effective amount of a 08K-3β inhibitor such as 3-(5-fluorobenzofuran-3-y1)-4-(5-methyl-5H-[1,3]dioxolo[4,5-f]indol-7-y1)pynOle-2,5-dione, or a pharmaceutically acceptable salt thereof, optionally in combination with a therapeutically effective amount of a JAK inhibitor such as ruxolitinib, or a pharmaceutically acceptable salt thereof.

### CROSS REFERENCE TO RELATED APPLICATION

This application claims the benefit of U.S. Provisional Pat. Application No. 62/953,654, filed on Dec. 26, 2019, entitled “COMPOUNDS FOR THE TREATMENT OF MYELOFIBROSIS,” the contents of which are hereby incorporated by reference herein.

### TECHNICAL FIELD OF THE INVENTION

The present invention relates to methods of treating myelofibrosis with a GSK-3β inhibitor such as 3-(5-fluorobenzofuran-3-yl)-4-(5-methyl-5H-[1,3]dioxolo[4,5-f]indol-7-yl)pyrrole-2,5-dione, optionally in combination with a JAK inhibitor such as ruxolitinib.

### BACKGROUND OF THE INVENTION

Myelofibrosis (MF) is lethal due to a mixture of true malignancy and marrow fibrosis excess. Although JAK2 inhibitors provide substantial clinical benefit, their disease-modifying activity is limited, and rational combinations with other targeted agents are needed, particularly in MF, in which survival is short.

Accordingly, there remains a need for improved treatment of MF.

### SUMMARY OF THE INVENTION

The compound 3-(5-fluorobenzofuran-3-yl)-4-(5-methyl-5H-[1,3]dioxolo[4,5-f]indol-7-yl)pyrrole-2,5-dione (hereinafter “9-ING-41”) is a small molecule and potent selective GSK-3β inhibitor with antitumor activity (Pal 2014, Ugolkov 2016, Ugolkov 2017). It acts through downregulation of NF-ₖB and decreases the expression NF-ₖB target genes cyclin D1, Bcl-2, anti-apoptotic protein (XIAP) and B-cell lymphoma-extra large (Bcl-XL) leading to inhibition of tumor growth in multiple solid tumor cell and lymphoma lines and patient derived xenograft (PDX) models. NF-ₖB is constitutively active in cancer cells and promotes anti-apoptotic molecule expression. NF-ₖB activation is particularly important in cancer cells that have become chemo- and radioresistant, therefore it is believed that inhibition of GSK-3β may overcome NF-kB-mediated chemoresistance in human cancers.

It has been found that 9-ING-41 is useful in treating certain forms of cancer, such as myelofibrosis.

Accordingly, in one aspect, the present invention provides a method of treating myelofibrosis in a patient by administering to the patient a therapeutically effective amount of a GSK-3β inhibitor such as 9-ING-41, or a pharmaceutically acceptable salt thereof.

Accordingly, in another aspect, the present invention provides a method of treating myelofibrosis in a patient by administering to the patient a therapeutically effective amount of a GSK-3β inhibitor such as 9-ING-41, or a pharmaceutically acceptable salt thereof, in combination with a therapeutically effective amount of a JAK inhibitor such as ruxolitinib or fedratinib, or a pharmaceutically acceptable salt thereof.

### BRIEF DESCRIPTION OF FIGURES

FIGS. 1A-1I depict hematopoietic colony growth frequency, plotted by colony type relative to % DMSO (no treatment) with 9-ING-41 alone or in combination with ruxolitinib (at 0.05 µM). FIG. 1A depicts MF Case 1: 9-ING-41 only. FIG. 1B depicts MF Case 1: 9-ING-41 + ruxolitinib. FIG. 1C depicts MF Case 1: ruxolitinib only. FIG. 1D depicts MF Case 2: 9-ING-41 only. FIG. 1E depicts MF Case 2: 9-ING-41 + ruxolitinib. FIG. 1F depicts MF Case 2: ruxolitinib only. FIG. 1G depicts Normal Bone Marrow (BM): 9-ING-41 only. FIG. 1H depicts Normal BM: 9-ING-41 + ruxolitinib. FIG. 1I depicts Normal BM: ruxolitinib only. Colony types are as follows: CFU-GM = granulocytic/monocyte(gray), CFU-G=granulocyte(black), BFU-E=erythroid(red), GEMM = primitive granulocytic/erythroid/ macrophage/monocyte(blue). Data are represented as percent of no treatment (DMSO only) and plotted by colony type and treatment. Error bars indicate standard deviation. Data from Terra Lasho, Mayo Clinic, Rochester, MN.

FIGS. 2A-2F depict GEMM colony frequency comparison (by % GEMM in DMSO) with 9-ING-41 only and in combination with sub-therapeutic levels of ruxolitinib (0.05 µM) in MF (n=2) and normal bone marrow (n=1). Colonies are plotted as percentage of no treatment (DMSO). FIG. 2A depicts MF Case 1: DMSO; ruxolitinib only; 9-ING-41 only; and 9-ING-41 + ruxolitinib. FIG. 2B depicts MF Case 1: representative colony morphology for DMSO; ruxolitinib only; 9-ING-41 only; and 9-ING-41 + ruxolitinib, respectively. FIG. 2C depicts MF Case 2: DMSO; ruxolitinib only; 9-ING-41 only; and 9-ING-41 + ruxolitinib. FIG. 2D depicts MF Case 2: representative colony morphology for DMSO; ruxolitinib only; 9-ING-41 only; and 9-ING-41 + ruxolitinib, respectively. FIG. 2E depicts Normal Bone Marrow (BM): DMSO; ruxolitinib only; 9-ING-41 only; and 9-ING-41 + ruxolitinib. FIG. 2F depicts MF Case 2: representative colony morphology for DMSO; ruxolitinib only; 9-ING-41 only; and 9-ING-41 + ruxolitinib, respectively. Photo size represents a 2 mm x 2 mm area. Data from Terra Lasho, Mayo Clinic, Rochester, MN.

### DETAILED DESCRIPTION OF THE INVENTION

### 1. General Description of Certain Embodiments of the Invention

Myelofibrosis (MF) is a myeloproliferative neoplasm characterized by ineffective clonal haematopoiesis, splenomegaly, bone marrow fibrosis, and the propensity for transformation to acute leukemia (Scheiber 2019). The discovery of mutations in JAK2, CALR, and MPL have focused on activated JAK-STAT signaling as a primary driver of MF. Two JAK inhibitors have been approved by the FDA for the treatment of patients with advanced MF. However, JAK inhibition alone is insufficient for long-term remission and offers modest, if any, sustained disease-modifying effects in the great majority of patients. Achieving sustained adequate exposure to the JAK inhibitors is a key factor for optimal therapeutic outcomes but adverse events, particularly myelosuppression, lead to dose reductions or interruptions in the majority of patients. Thus, anti-neoplastic agents with modes of action that are independent of direct JAK-STAT inhibition are of particular interest as are agents that may resolve pathologic fibrosis - an approach that has recently proven to be of clinical value in MF (Verstovsek 2015).

Glycogen synthase kinase-3 (GSK-3) is a serine (S)/threonine (T)(ST) kinase initially described as a key regulator of metabolism, specifically glycogen biosynthesis (Woodgett 1990). It has since been shown to play a role in several disease processes including cancer, immune disorders, metabolic disorders, pleural fibrosis and neurological disorders through modulation of a large and diverse number of substrates (Boren 2017, Farghaian 2011, Gao 2011, Wang 2011a, Klamer 2010, Henriksen 2010). GSK-3 has two ubiquitously expressed and highly conserved isoforms, glycogen synthase kinase-3 alpha (GSK-3α) and glycogen synthase kinase-3 beta (GSK-3β), with both shared and distinct substrates and functional effects. GSK-3 is found in all eukaryotes. It is a key regulator of numerous signaling pathways, including cellular responses to Wnt, G protein-coupled receptors and receptor tyrosine kinases. GSK-3 is usually constitutively active in cells and is regulated through inhibition of its activity. As distinct from other protein kinases, GSK-3's prefers primed substrates, i.e. substrates previously phosphorylated by another kinase (Doble 2003).

In cancer, much focus has been placed on the role of GSK-3β in tumor progression and its modulation of oncogenes (beta-catenin, cyclin D1 and c-Myc), cell cycle regulators (e.g. p27Kip1) and mediators of epithelial-mesenchymal transition (e.g. Zinc finger protein SNAI1, Snail) have been extensively described (Doble 2007, Gregory 2003, An 2008, Lin 2000, Wang 2013). More recently, aberrant overexpression of GSK-3β has been shown to promote tumor growth and chemotherapy resistance in various solid tumors including pancreatic, ovarian, colon cancer and glioblastoma (Ougolkov 2005, Fu 2011, Shakoori 2005, Mai 2009, Miyashita 2009a) through differential effects on pro-survival nuclear factor kappa-light-chain-enhancer of activated B cells (NF-kB) and c-Myc pathways as well on tumor necrosis factor (TNF)-related apoptosis-inducing ligand (TRAIL) and p53-mediated apoptotic mechanisms (Liao 2003, Tan 2005). GSK-3β is thus a potentially very important therapeutic target in human malignancies.

While GSK-3 is an ST protein kinase originally described as a key enzyme involved in glycogen metabolism (Woodgett 1990, Welsh 1993), it is now also known to regulate a diverse array of cellular functions ranging from glycogen metabolism to cell-cycle regulation and proliferation (Cohen 2001). GSK-3 exerts its function by phosphorylating and thereby regulating the function of many metabolic, signaling, and structural proteins (Cohen 2001). It has also been implicated in the pathogenesis of various human diseases, including type II diabetes, Alzheimer’s disease, bipolar disorder, inflammation, pleural fibrosis and cancer (Boren 2017, Pal 2014). There are two highly homologous forms of GSK-3 in mammals, GSK-3-α and GSK-3β (Cohen 2001) both exhibiting kinase activity (Woodgett 1990). Although historically GSK-3β has been thought of as a potential tumor suppressor due to its ability to phosphorylate pro-oncogenic molecules e.g. c-Myc (Sears 2000), cyclin D1 (Diehl 1998) and β-catenin (Hart 1998), thereby targeting these molecules for ubiquitin-proteasome degradation, recent reports have suggested that GSK-3 is a positive regulator of cancer cell proliferation and survival (Wang 2011a, Wang 2013, Ougolkov 2005, Shakoori 2005, Pal 2014, Bilim 2009, Cao 2006a, Carter 2014, Dickey 2011, Duffy 2014, Gaisina 2009, Hilliard 2011, Kotliarova 2008, Kunnimalaiyaan 2007, Miyashita 2009b, Naito 2010, Ougolkov 2006a, Ougolkov 2008, Ougolkov 2007, Ougolkov 2006b, Shin 2014, Wang 2011, Wang 2011b, Wang 2009, Wang 2008, Zeng 2014, Zhu 2011) and has prompted the development of GSK-3 specific inhibitors as therapeutic targets.

GSK-3β was previously described as a potential anticancer target in human pancreatic, colon, bladder and renal cancer cells, and chronic lymphocytic leukemia (Shakoori 2005, Bilim 2009, Gaisina 2009, Naito 2010, Ougolkov 2006a, Ougolkov 2007). Recent studies demonstrated that GSK-3β is also a promising therapeutic target in glioblastoma, neuroblastoma, thyroid, ovarian, colorectal, lung and prostate cancer (Miyashita 2009a, Pal 2014, Carter 2014, Dickey 2011, Duffy 2014, Hilliard 2011, Kotliarova 2008, Kunnimalaiyaan 2007, Shin 2014, Wang 2009, Zeng 2014, Zhu 2011, Cao 2006b). The potent maleimide-based GSK-3β inhibitor, 9-ING-41, was identified as a candidate for targeted therapy in chemoresistant human breast cancer (Ugolkov 2016). Its antiproliferative activity involves G0-G1 and G2-M phase arrest, a mechanism evident from cell-cycle analysis in renal cell carcinoma cell lines (Pal 2014).

NF-kB is regarded as one of the most important transcription factors and its activation plays an essential role in promoting human cancer progression, metastasis, and chemoresistance (Aggarwal 2004, Tas 2009). GSK-3β has been demonstrated to have opposing roles in this context, repressing Wnt/beta-catenin signaling on the one hand but maintaining cell survival and proliferation through the NF-kB pathway on the other (Shakoori 2005). Recent data suggests that GSK-3β positively regulates human cancer cell survival in part through regulation of NF-κB-mediated expression of anti-apoptotic molecules (Bilim 2009). Disruption of the GSK-3β gene in mice leads to embryonic lethality due to hepatocyte apoptosis and massive liver degeneration, a phenotype that is similar to the disruption of the NF-kB p65 or inhibitor of nuclear factor kappa-B kinase subunit beta (IKKβ) genes (Hoeflich 2000). These findings suggest a link between GSK-3β and the activation of the NF-kB pathway and support GSK-3β as a candidate therapeutic target in human cancer.

9-ING-41 is a small molecule potent selective GSK-3β inhibitor with antitumor activity (Pal 2014, Ugolkov 2016, Ugolkov 2017). It acts through downregulation of NF-κB and decreases the expression NF-kB target genes cyclin D1, Bcl-2, anti-apoptotic protein (XIAP) and B-cell lymphoma-extra large (Bcl-XL) leading to inhibition of tumor growth in multiple solid tumor cell and lymphoma lines and patient derived xenograft (PDX) models. NF-kB is constitutively active in cancer cells and promotes anti-apoptotic molecule expression. NF-kB activation is particularly important in cancer cells that have become chemo- and radioresistant, therefore it is believed that inhibition of GSK-3β may overcome NF-kB-mediated chemoresistance in human cancers.

It has been found that 9-ING-41 is useful in treating certain forms of cancer, such as myelofibrosis.

In some embodiments, the present invention provides a method for treating myelofibrosis in a patient, comprising administering to the patient a therapeutically effect amount of a GSK-3β inhibitor such as 9-ING-41, or a pharmaceutically acceptable salt thereof.

In some embodiments, the present invention provides a GSK-3β inhibitor such as 9-ING-41, or a pharmaceutically acceptable salt thereof, for use in treating myelofibrosis.

In some embodiments, the present invention provides for the use of a GSK-3β inhibitor such as 9-ING-41, or a pharmaceutically acceptable salt thereof, to treat myelofibrosis.

In some embodiments, the present invention provides for the use of a GSK-3β inhibitor such as 9-ING-41, or a pharmaceutically acceptable salt thereof, in the preparation of a medicament for use in treating myelofibrosis.

In some embodiments, the present invention provides a method for treating solid tumors in a patient, comprising administering to the patient a therapeutically effect amount of a GSK-3β inhibitor such as 9-ING-41, or a pharmaceutically acceptable salt thereof, in combination with a JAK inhibitor such as ruxolitinib, or a pharmaceutically acceptable salt thereof.

In some embodiments, the present invention provides a pharmaceutical combination comprising a GSK-3β inhibitor such as 9-ING-41, or a pharmaceutically acceptable salt thereof, and a JAK inhibitor such as ruxolitinib, or a pharmaceutically acceptable salt thereof, for use in treating myelofibrosis.

In some embodiments, the present invention provides for the use of a pharmaceutical combination comprising a GSK-3β inhibitor such as 9-ING-41, or a pharmaceutically acceptable salt thereof, and a JAK inhibitor such as ruxolitinib, or a pharmaceutically acceptable salt thereof, to treat myelofibrosis.

In some embodiments, the present invention provides for the use of a GSK-3β inhibitor such as 9-ING-41, or a pharmaceutically acceptable salt thereof, in the preparation of a medicament for use in treating myelofibrosis, in combination with a JAK inhibitor such as ruxolitinib, or a pharmaceutically acceptable salt thereof.

In some embodiments, the present invention provides a kit comprising 9-ING-41, or a pharmaceutically acceptable salt thereof, and ruxolitinib, or a pharmaceutically acceptable salt thereof.

In some embodiments, the present invention provides a kit comprising 9-ING-41, or a pharmaceutically acceptable salt thereof, and ruxolitinib, or a pharmaceutically acceptable salt thereof, for use in treating myelofibrosis.

In some embodiments, the present invention provides for the use of a kit comprising 9-ING-41, or a pharmaceutically acceptable salt thereof, and ruxolitinib, or a pharmaceutically acceptable salt thereof, to treat myelofibrosis.

### 2. Definitions

As used herein, “9-ING-41” refers to 3-(5-fluorobenzofuran-3-yl)-4-(5-methyl-5H-[1,3]dioxolo[4,5-f]indol-7-yl)pyrrole-2,5-dione, having the structure:

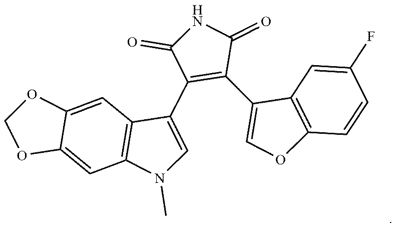

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient,” as used herein, means an animal, preferably a mammal, and most preferably a human, preferably at least 18 years old.

### 3. Description of Exemplary Methods and Uses

In some embodiments, the present invention provides a method of treating myelofibrosis in a patient, comprising administering to the patient a therapeutically effect amount of a glycogen synthase kinase-3 beta (GSK-3β) inhibitor, or a pharmaceutically acceptable salt thereof. In some embodiments, the GSK-3β inhibitor is 9-ING-41, or a pharmaceutically acceptable salt thereof.

In some embodiments, the GSK-3β inhibitor, or pharmaceutically acceptable salt thereof, is administered to the patient in a range of from about 1 mg/kg to about 50 mg/kg. In some embodiments, the GSK-3β inhibitor, or pharmaceutically acceptable salt thereof, is administered to the patient in a range of from about 5 mg/kg to about 15 mg/kg. In some embodiments, about 9 mg/kg of the GSK-3β inhibitor, or pharmaceutically acceptable salt thereof, is administered to the patient.

In some embodiments, the GSK-3β inhibitor, or pharmaceutically acceptable salt thereof, is administered to the patient once per week during a 28-day treatment cycle. In some embodiments, the GSK-3β inhibitor, or pharmaceutically acceptable salt thereof, is administered to the patient twice per week during a 28-day treatment cycle. In some embodiments, the GSK-3β inhibitor, or pharmaceutically acceptable salt thereof, is administered to the patient on days 1 and 4 of the week. In some embodiments, the GSK-3β inhibitor, or pharmaceutically acceptable salt thereof, is administered to the patient intravenously.

In some embodiments, the method of treating myelofibrosis further comprises administering to the patient a therapeutically effect amount of a JAK inhibitor, or a pharmaceutically acceptable salt thereof. In some embodiments, the JAK inhibitor is selected from the group consisting of pacritinib, momelotinib, fedratinib and ruxolitinib, or a pharmaceutically acceptable salt thereof. In some embodiments, the JAK inhibitor is ruxolitinib, or a pharmaceutically acceptable salt thereof.

In some embodiments, the JAK inhibitor, or pharmaceutically acceptable salt thereof, is administered to the patient in a range of from about 1 mg to about 50 mg. In some embodiments, the JAK inhibitor, or pharmaceutically acceptable salt thereof, is administered to the patient in amounts of: about 5 mg twice a day for patients with a platelet count ≥ 20,000/mL; or about 10 mg twice day for patients with a platelet count ≥50,000/mL; or about 15 mg twice a day for patients with platelet count ≥100,000/mL; or about 20 mg twice a day for patients with platelet count ≥200,000/mL.

In some embodiments, the JAK inhibitor, or pharmaceutically acceptable salt thereof, is administered to the patient twice per day during a 28-day treatment cycle. In some embodiments, the JAK inhibitor, or pharmaceutically acceptable salt thereof, is administered to the patient orally.

In some embodiments, the present invention provides a method for treating myelofibrosis in a patient, comprising administering to the patient a therapeutically effect amount of 9-ING-41, or a pharmaceutically acceptable salt thereof.

In some embodiments, the present invention provides a method of treating myelofibrosis in a patient, comprising administering to the patient a therapeutically effect amount of a glycogen synthase kinase-3 Beta(GSK-3β) inhibitor, or a pharmaceutically acceptable salt thereof, in combination with a therapeutically effect amount of a JAK inhibitor, or a pharmaceutically acceptable salt thereof. In some embodiments, the GSK-3β inhibitor is 9-ING-41, or a pharmaceutically acceptable salt thereof.

In some embodiments, the GSK-3β inhibitor, or pharmaceutically acceptable salt thereof, is administered to the patient in a range of from about 1 mg/kg to about 50 mg/kg. In some embodiments, the GSK-3β inhibitor, or pharmaceutically acceptable salt thereof, is administered to the patient in a range of from about 5 mg/kg to about 15 mg/kg. In some embodiments, about 9 mg/kg of the GSK-3β inhibitor, or pharmaceutically acceptable salt thereof, is administered to the patient.

In some embodiments, the GSK-3β inhibitor, or pharmaceutically acceptable salt thereof, is administered to the patient once per week during a 28-day treatment cycle. In some embodiments, the GSK-3β inhibitor, or pharmaceutically acceptable salt thereof, is administered to the patient twice per week during a 28-day treatment cycle. In some embodiments, the GSK-3β inhibitor, or pharmaceutically acceptable salt thereof, is administered to the patient on days 1 and 4 of the week. In some embodiments, the GSK-3β inhibitor, or pharmaceutically acceptable salt thereof, is administered to the patient intravenously.

In some embodiments, the JAK inhibitor is selected from the group consisting of pacritinib, momelotinib, fedratinib and ruxolitinib, or a pharmaceutically acceptable salt thereof. In some embodiments, the JAK inhibitor is ruxolitinib, or a pharmaceutically acceptable salt thereof.

In some embodiments, the JAK inhibitor, or pharmaceutically acceptable salt thereof, is administered to the patient in a range of from about 1 mg to about 50 mg. In some embodiments, the JAK inhibitor, or pharmaceutically acceptable salt thereof, is administered to the patient in amounts of: about 5 mg twice a day for patients with a platelet count ≥ 20,000/mL; or about 10 mg twice day for patients with a platelet count ≥50,000/mL; or about 15 mg twice a day for patients with platelet count ≥100,000/mL; or about 20 mg twice a day for patients with platelet count ≥200,000/mL.

In some embodiments, the JAK inhibitor, or pharmaceutically acceptable salt thereof, is administered to the patient twice per day during a 28-day treatment cycle. In some embodiments, the JAK inhibitor, or pharmaceutically acceptable salt thereof, is administered to the patient orally.

In some embodiments, the present invention provides a method of treating myelofibrosis in a patient, comprising administering to the patient a therapeutically effect amount of 9-ING-41, or a pharmaceutically acceptable salt thereof.

In some embodiments, 9-ING-41, or a pharmaceutically acceptable salt thereof, is administered to the patient in a range of from about 5 mg/kg to about 15 mg/kg. In some embodiments, about 9 mg/kg of 9-ING-41, or a pharmaceutically acceptable salt thereof, is administered to the patient. In some embodiments, 9-ING-41, or pharmaceutically acceptable salt thereof, is intravenously administered to the patient on days 1 and 4 of each week during a 28-day treatment cycle.

In some embodiments, the method of treating myelofibrosis further comprises administering to the patient a therapeutically effect amount of ruxolitinib, or a pharmaceutically acceptable salt thereof. In some embodiments, ruxolitinib, or pharmaceutically acceptable salt thereof, is orally administered to the patient in a range of from about 1 mg to about 50 mg. In some embodiments, ruxolitinib, or pharmaceutically acceptable salt thereof, is orally administered to the patient in amounts of: about 5 mg twice a day for patients with a platelet count ≥ 20,000/mL; or about 10 mg twice day for patients with a platelet count ≥50,000/mL; or about 15 mg twice a day for patients with platelet count ≥100,000/mL; or about 20 mg twice a day for patients with platelet count ≥200,000/mL.

In some embodiments, the present invention provides a method of treating myelofibrosis in a patient, comprising administering to the patient a therapeutically effect amount of 9-ING-41, or a pharmaceutically acceptable salt thereof, in combination with a therapeutically effect amount of ruxolitinib, or a pharmaceutically acceptable salt thereof.

In some embodiments, 9-ING-41, or pharmaceutically acceptable salt thereof, is administered to the patient in a range of from about 1 mg/kg to about 50 mg/kg. In some embodiments, 9-ING-41, or pharmaceutically acceptable salt thereof, is administered to the patient in a range of from about 5 mg/kg to about 15 mg/kg. In some embodiments, about 9 mg/kg of 9-ING-41, or pharmaceutically acceptable salt thereof, is administered to the patient. In some embodiments, 9-ING-41, or pharmaceutically acceptable salt thereof, is intravenously administered to the patient on days 1 and 4 of each week during a 28-day treatment cycle.

In some embodiments, ruxolitinib, or pharmaceutically acceptable salt thereof, is orally administered to the patient in amounts of: about 5 mg twice a day for patients with a platelet count ≥20,000/mL; or about 10 mg twice day for patients with a platelet count ≥ 50,000/mL; or about 15 mg twice a day for patients with platelet count ≥100,000/mL; or about 20 mg twice a day for patients with platelet count ≥200,000/mL.

In some embodiments, the present invention provides a kit comprising 9-ING-41, or a pharmaceutically acceptable salt thereof, and ruxolitinib, or a pharmaceutically acceptable salt thereof. In some embodiments, the kit comprises a set of instructions for using the kit in a method of treating myelofibrosis. In some embodiments, the set of instructions provided in the kit suitably can be written, such as on paper, or on the kit packaging, or otherwise provided as a link to a websites' address or suitable code, such as a QR code, for looking up the instructions on the internet.

In some embodiments, a tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

The following examples are provided for illustrative purposes only and are not to be construed as limiting this invention in any manner.

### EXEMPLIFICATION

Compounds useful in the method of the present invention include-9-ING-41, described in US Pat. 8,207,216 (Kozikowski et al.), incorporated by reference in its entirety.

### Example 1 - Ex Vivo Studies of 9-ING-41 as a Single Agent or Combined with Ruxolitinib on Growth and Proliferation of Myelofibrosis Cells

In order to study the effects of 9-ING-41 (alone and in combination with Ruxolitinib) on growth and proliferation in MF, ex vivo colony assays were conducted using primary cells from patients with previously untreated MF and normal bone marrow. The experiments were designed to assess the number, size, and morphology of stem/progenitor cells present in blood both with and without treatment.

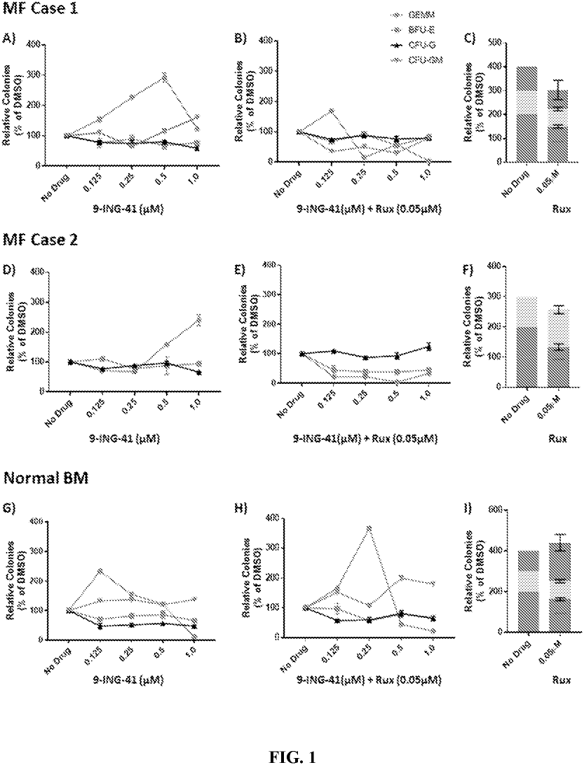

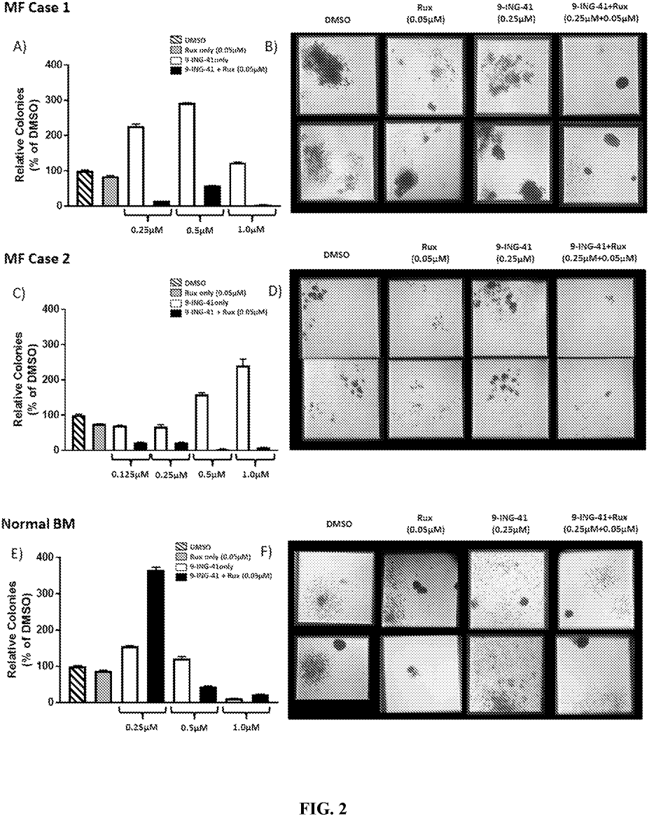

Peripheral blood mononuclear cells from MF patients and bone marrow from healthy patients (control) were plated in methylcellulose in duplicate, containing cytokines, in the presence of either DMSO only, 9-ING-41 only, or a combination of 9-ING-41 and Ruxolitinib (0.05uM). Colonies were counted ten days later, and colony growth frequency, distribution, and morphology were calculated. The presence of erythroid (BFU-E), granulocytic (CFU-G), and granulocytic/monocytic (CFU-GM) colonies remained proportionally the same after addition of 9-ING-41, whereas the more primitive granulocytic/erythroid/macrophage/monocyte (GEMM) colony growth increased relative to 9-ING-41 concentration in MF cases as compared to normal, as shown in FIG. 1 (e.g., FIGS. 1A, 1D and 1G) and FIG. 2 (e.g., FIGS. 2A, 2C and 2E).

This suggests a selective primitive proliferative and/or differentiation effect of GSK3β inhibition by 9-ING-41. Combinatory treatment of 9-ING-41 with Ruxolitinib (at a sub-therapeutic level -50 nM) appeared to erase this effect in a dose dependent manner, as shown in FIG. 1 (e.g., FIGS. 1B, 1E and 1H) and FIG. 2 (e.g., FIGS. 2A, 2C and 2E). The comparison of effect on colony size using sub-therapeutic concentrations of 0.05 µM Ruxolitinib alone (for combinatory experiments) is shown in FIG. 1 (e.g., FIGS. 1C, 1F and 1I).

Surprisingly, morphology of the colonies in treatments without 9-ING-41 was observed to be markedly different than morphology of the colonies in treatments with 9-ING-41. In MF cases 1 and 2, with no treatment (DMSO) and addition of Ruxolitinib only (0.05 µM), the colonies appeared similar to each other and were irregular and disorganized relative to normal bone marrow, as shown in FIG. 2 (e.g., FIGS. 2B and 2D). When cells were treated with 9-ING-41 alone, there was an increase in very large, fully differentiated primitive colonies in MF cases 1 and 2, and a modest increase in colony size in normal marrow was observed. With the combination of Ruxolitinib with 9-ING-41, in MF cases 1 and 2 there was a significant decrease in the size and presence of primitive colonies, and the appearance of normal, healthy-looking colonies (discreet, round size and relative to normal bone marrow colonies).

### Example 2 - A Phase 2 Study of 9-ING-41 as a Single Agent or Combined with Ruxolitinib, in Patients with Myelofibrosis

### Objectives

Primary: To evaluate the efficacy of 9-ING-41 as monotherapy and in combination with ruxolitinib in patients with myelofibrosis.

Secondary:
1) To evaluate the effect of 9-ING-41 on bone marrow fibrosis;
2) To evaluate the effect of 9-ING-41 on spleen volume;
3) To evaluate the effect of 9-ING-41 on anemia;
4) To evaluate the effect of 9-ING-41 on total symptom score (TSS) as assessed by the Myelofibrosis Symptom Assessment Form (MFSAF) version 4.0 diary; and
5) To assess pharmacokinetics and pharmacodynamics of 9-ING-41.

Exploratory objectives include a) quality of life measured by EORTC QLQ-C30 questionnaire; b) allelic burden (JAK2V617F, calreticulin [CALR], MPLW515L/K); c) cytogenetic response; d) inflammatory cytokines measurements; and e) flow cytometry of peripheral blood.

### Endpoints

The efficacy endpoints are the following:
1) Response rate (RR), defined as the percent of patients with Complete Response (CR), Partial Response (PR) or Clinical improvement (CI) according to the Revised IWG-MRT and ELN Response Criteria for MF (2013);
2) Duration of Response (DoR), defined as the time from documentation of tumor response to disease progression;
3) Progression-Free Survival (PFS), defined as the time from study enrolment until objective tumor progression or death; and
4) Overall survival (OS), defined as the time from study entry to death from any cause

Time-to event endpoints (DoR, PFS, and OS) will be summarized by Kaplan-Meier methods (median, 95% CI, number of events, number censored and Kaplan-Meier figures). Adverse events will be monitored during the period starting on the date of patient signature of study informed consent form and ending 30 days after the final administration of 9-ING-41. All patients who receive any dose (any amount) of 9-ING-41 will be included in the summaries and listings of safety data. Overall safety profile and tolerability will be characterized by type, frequency, severity, timing, duration and relationship of study drug to adverse events and laboratory abnormalities.

### Study Design

This is an open label, multi-center non-randomized Phase 2 study of 9-ING-41 as a single agent or when combined with ruxolitinib in patients with advanced myelofibrosis. Treatment will consist of twice-weekly intravenous (IV) infusion of 9-ING-41 as a single agent or in combination with ruxolitinib.

### Study Population/Patient Eligibility - Inclusion Criteria

Patient must meet ALL the following criteria to be eligible for this study:
1) Is able to understand and voluntarily sign a written informed consent and is willing and able to comply with the protocol requirements including scheduled visits, treatment plan, laboratory tests and other study procedures;
2) Is aged ≥ 18 years;
3) Has documented diagnosis of primary MF, PPV-MF or PET-MF as defined by the World Health Organization classification with a DIPSS plus score of ≥4;
4) Is ineligible or unwilling to undergo stem cell transplantation at time of study entry;
5) Has laboratory function within specified parameters per local laboratory reference range (may be repeated):
   Absolute neutrophil count (ANC) ≥100/mL; platelets ≥20,000/mL
   Transaminases (AST/ALT) and alkaline phosphatase ≤ 3 (≤ 10 X the upper limit of normal (ULN) if considered to be MF-related) x ULN; bilirubin ≤ 1.5 x ULN (unless patient has Gilbert’s Syndrome)
   Serum amylase and lipase ≤ 1.5 x ULN;
6) Has adequate performance status (PS): Eastern Co-operative Oncology Group (ECOG) PS 0-2;
7) Has received the final dose of any of the following treatments/procedures with the specified minimum intervals before first dose of 9-ING-41 (unless in the opinion of the investigator and the study medical coordinator the treatments/procedures will not compromise patient safety or interfere with study conduct:
   Chemotherapy, immunotherapy, or systemic radiation therapy, 14 days maximum, or ≥ 5 half-lives (whichever is shorter
   Surgery with general anesthesia - 7 days;
8) Patients who are to receive 9-ING-41 plus Ruxolitinib must have attempted ≥12 weeks of Ruxolitinib therapy and required dose reductions/interruptions and/or had an inadequate response. Patients with overtly progressive disease may be enrolled on study with less than a 12-week duration of attempted Ruxolitinib therapy by agreement between the investigator and the study medical coordinator.
9) Women of childbearing potential must have a negative baseline blood or urine pregnancy test within 72 hours of first study therapy. Women may be neither breastfeeding nor intending to become pregnant during study participation and must agree to use effective contraceptive methods (hormonal or barrier method of birth control, or true abstinence) for the duration of study participation and in the following 100 days after discontinuation of study treatment.
10) Male patients with partners of childbearing potential must take appropriate precautions to avoid fathering a child from screening until 100 days after discontinuation of study treatment and use appropriate barrier contraception or true abstinence.
11) Must not be receiving any other investigational product

### Study Population/Patient Eligibility - Exclusion Criteria

Patients who meet any of the following criteria are not eligible for this study:
1) Is pregnant or lactating;
2) Is known to be hypersensitive to any of the components of 9-ING-41 or to the excipients used in its formulation;
3) Has >10% blasts in peripheral blood or bone marrow biopsy;
4) Has had a myocardial infarction within 12 weeks of the first dose of 9-ING-41;
5) Has any medical and/or social condition which, in the opinion of the investigator or study medical coordinator would preclude study participation;
6) Is considered to be a member of a vulnerable population (for example, prisoners); or
7) Herbal preparations / medications are prohibited throughout the study. These herbal medications include, but are not limited to St. John’s wort, Kava, ephedra (ma huang), Gingko biloba, dehydroepiandrosterone (DHEA), yohimbe, saw palmetto, and Ginseng. Patients should stop using cannabinoids or herbal preparations / medications at least 7 days prior to first dose of study treatment.

### Administration of 9-ING-41

9-ING-41 will be administered on Day 1 and 4 of each week of a 28-day cycle at a dose of 9.3 mg/kg, either as a single agent or in combination with Ruxolitinib.[00103] All patients should be weighed within 72 hours prior to dosing for every cycle to ensure they did not experience either a weight loss or gain >10% from the prior weight used to calculate the dose of 9-ING-41. The decision to recalculate dose(s) according to a change in weight should be in accordance with local practice, however where weight has changed by >10%, dose MUST be recalculated using the most recent weight recorded.

### Administration of 9-ING-41 + Ruxolitinib

9-ING-41 9.3 mg/kg will be administered by intravenous infusion twice weekly on days 1 and 4 for cycle durations of 28 days with Ruxolitinib at last prior tolerated dose with minimum of:
5 mg PO twice daily for patients with platelet count ≥20,000/mL;
10 mg PO twice daily for patients with platelet count ≥50,000/mL;
15 mg PO twice daily for patients with platelet count ≥100,000/mL; or
20 mg PO twice daily for patients with platelet count ≥200,000/mL.

If patients have baseline Grade ¾ anemia with platelet count ≥50,000/mL, the initial Ruxolitinib dose may be reduced by 5 mg PO twice daily. If the last tolerated Ruxolitinib dose immediately prior to study entry was less than above, the initial on-study dose of Ruxolitinib may be reduced to that dose after discussion with medical monitor.

After each cycle of therapy, if the response is considered inadequate, the Ruxolitinib dose may be increased in 5 mg PO twice daily increments to a maximum of 25 mg PO twice daily. When discontinuing therapy for any reason other than thrombocytopenia, consider gradually tapering Ruxolitinib dose by 5 mg twice daily each week.

Patients will continue study drug regimen for as long as the patient does not have clinically significant progressive disease and/or unacceptable toxicity and as long as the investigator deems that the patient is benefiting from treatment. Treatment may also be stopped if the patient withdraws consent, or study termination occurs (see Section 2.7.1).

### Safety Evaluation

Safety will be assessed throughout the study including by recording and monitoring Adverse events (AEs) (CTCAE v5), vital signs (blood pressure, pulse, respiratory rate, and body temperature), physical examination findings, serum chemistry and hematology laboratory values, urinalysis, ECG, and concomitant medication usage. Aside from those detailed in study assessment schedule, relevant assessments consistent with best patient care should be performed and recorded on the study case record forms.

### Efficacy Evaluation

Response will be evaluated as per the 2013 Revised IWG-MRT and ELN Response Criteria for MF. Only evaluable patients will be considered for efficacy measurement. All patients who have received at least one cycle of 9-ING-41 therapy will be considered evaluable for response. Aside from those detailed in the study assessment schedule, relevant assessments consistent with best patient care should be performed and recorded on the study case record forms where appropriate.

Standard of care assessments will be performed during screening, treatment and follow-up until disease progression is documented, patient initiates new anticancer therapy, patient withdraws their consent to study participation, or patient completes a 12-month follow-up period after the last dose of study drug, whichever occurs first. Patients with a documented response will be required to have an assessment 4-8 weeks later to confirm the response as per standard of care.

### Statistical Considerations

A Simon 2-Stage optimal model will be used for enrollment based on efficacy.

For single agent 9-ING-41 therapy, up to 10 fully evaluable patients will be treated and if there are no patients with a response, the study arm will be closed. Otherwise, 19 additional fully evaluable patients will be accrued for a total of 29 patients. If 4 or more responses are observed in 29 patients, the conclusion will be that the regimen is worthy of further investigation. When the response rate of interest of 20% (alternative hypothesis) is tested against the null hypothesis response rate of 5%; this design yields a Type I error rate of 0.05 and power of 80%.

For 9-ING-41 plus Ruxolitinib therapy, up to 10 fully evaluable patients will be treated and if there are no patients with a response, the study arm will be closed. Otherwise, 19 additional fully evaluable patients will be accrued for a total of 29 patients. If 4 or more responses are observed in 29 patients, the conclusion will be that the regimen is worthy of further investigation. When the response rate of interest of 20% (alternative hypothesis) is tested against the null hypothesis response rate of 5%; this design yields a Type I error rate of 0.05 and power of 80%.

The proportion of patients with or without response will be tabulated with baseline levels of molecular, cytogenic and other biomarkers that may be assessed for signal as potential diagnostic or prognostic characteristics. As this is an open-label Phase 2 oncology study, descriptive statistics will be utilized for all safety and pharmacokinetic parameters. Categorical variables will be summarized by frequency distributions (number and percentages of patients), continuous variables will be summarized by mean, standard deviation, median, minimum, maximum, and time-to-event variables will be summarized using Kaplan-Meier methods and figures for the estimated median time. The primary objective is to assess efficacy as assessed by the rate of response. DoR, PFS and OS will also be assessed, and these time-to event endpoints summarized by Kaplan-Meier methods (median, 95% CI, number of events, number censored and Kaplan-Meier figures).

Frequencies of patients experiencing at least one AE will be displayed by body system and preferred term according to Medical Dictionary for Regulatory Activities (MedDRA) terminology. Detailed information collected for each AE will include description of the event, event duration, whether the AE was serious, severity, relationship to study drug, action taken, clinical outcome, and whether or not it was a DLT. Severity of the AEs will be graded according to the CTCAE v5. AEs classified as dose limiting will be listed.

Vital signs and ECGs will be summarized using descriptive statistics. Summary tables will be prepared to examine the distribution of laboratory measures over time. Shift tables may be provided to examine the distribution of laboratory toxicities.

Patient-reported symptomatic burden of disease is recorded using the Myeloproliferative Neoplasm Symptom Assessment Form Total Symptom Score (MPN-SAF-TSS). The patient records level of difficulty from 0 to 10 (0 for no difficulty and progressive difficulty up to 10 as worst imaginable) for each of 10 symptoms in the past week prior to screening/baseline and start of dosing cycle captures fatigue levels. Patients report fatigue level in the past 24 hours prior to each visit with 0 as no fatigue and progressively worse with 10 as worst. Descriptive statistics will be reported for each of the scores and for the total score by cohort and visit. Descriptive statistics for change from baseline at each visit will be reported by cohort.

The proportion of patients with specific biomarkers reported as binary or ordered categories will be reported by cohort. Non-parametric correlations between biological biomarkers and treatment responses will be reported.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

The present disclosure also encompasses the following aspects:

Aspect 1. A method of treating myelofibrosis in a patient, comprising administering to the patient a therapeutically effect amount of a glycogen synthase kinase-3 beta (GSK-3β) inhibitor, or a pharmaceutically acceptable salt thereof.

Aspect 2. The method of aspect 1, wherein the GSK-3β inhibitor is:

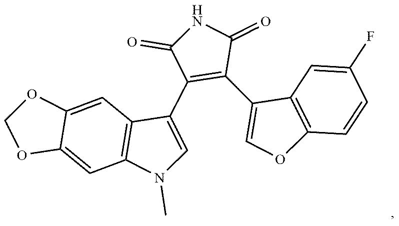

or a pharmaceutically acceptable salt thereof.

Aspect 3. The method of aspect 1, wherein the GSK-3β inhibitor, or pharmaceutically acceptable salt thereof, is administered to the patient in a range of from about 1 mg/kg to about 50 mg/kg.

Aspect 4. The method of aspect 1, wherein the GSK-3β inhibitor, or pharmaceutically acceptable salt thereof, is administered to the patient in a range of from about 5 mg/kg to about 15 mg/kg.

Aspect 5. The method of aspect 1, wherein about 9 mg/kg of the GSK-3β inhibitor, or pharmaceutically acceptable salt thereof, is administered to the patient.

Aspect 6. The method of aspect 1, wherein the GSK-3β inhibitor, or pharmaceutically acceptable salt thereof, is administered to the patient once per week during a 28-day treatment cycle.

Aspect 7. The method of aspect 1, wherein the GSK-3β inhibitor, or pharmaceutically acceptable salt thereof, is administered to the patient twice per week during a 28-day treatment cycle.

Aspect 8. The method of aspect 1, wherein the GSK-3β inhibitor, or pharmaceutically acceptable salt thereof, is administered to the patient on days 1 and 4 of the week.

Aspect 9. The method of aspect 1, wherein the GSK-3β inhibitor, or pharmaceutically acceptable salt thereof, is administered to the patient intravenously.

Aspect 10. The method of aspect 1, further comprising administering to the patient a therapeutically effect amount of a JAK inhibitor, or a pharmaceutically acceptable salt thereof.

Aspect 11. The method of aspect 10, wherein the JAK inhibitor is selected from the group consisting of pacritinib, momelotinib, fedratinib and ruxolitinib, or a pharmaceutically acceptable salt thereof.

Aspect 12. The method of aspect 10, wherein the JAK inhibitor is ruxolitinib, or a pharmaceutically acceptable salt thereof.

Aspect 13. The method of aspect 10, wherein the JAK inhibitor, or pharmaceutically acceptable salt thereof, is administered to the patient in a range of from about 1 mg to about 50 mg.

Aspect 14. The method of aspect 10, wherein the JAK inhibitor, or pharmaceutically acceptable salt thereof, is administered to the patient in amounts of:
about 5 mg twice a day for patients with a platelet count ≥20,000/mL; or
about 10 mg twice day for patients with a platelet count ≥50,000/mL; or
about 15 mg twice a day for patients with platelet count ≥100,000/mL; or
about 20 mg twice a day for patients with platelet count ≥200,000/mL.

Aspect 15. The method of aspect 10, wherein the JAK inhibitor, or pharmaceutically acceptable salt thereof, is administered to the patient twice per day during a 28-day treatment cycle.

Aspect 16. The method of aspect 10, wherein the JAK inhibitor, or pharmaceutically acceptable salt thereof, is administered to the patient orally.

Aspect 17. A method of treating myelofibrosis in a patient, comprising administering to the patient a therapeutically effect amount of a glycogen synthase kinase-3 Beta (GSK-3β) inhibitor, or a pharmaceutically acceptable salt thereof, in combination with a therapeutically effect amount of a JAK inhibitor, or a pharmaceutically acceptable salt thereof.

Aspect 18. The method of aspect 17, wherein the GSK-3β inhibitor is:

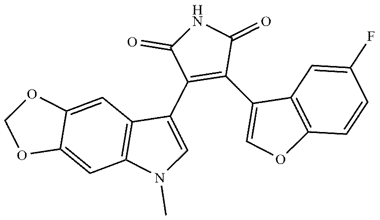 [SMILES: Cn1cc(C2=C(c3coc4ccc(F)cc34)C(=O)NC2=O)c2cc3c(cc21)OCO3]

ora pharmaceutically acceptable salt thereof. ,

Aspect 19. The method of aspect 17, wherein the JAK inhibitor is selected from the group consisting of pacritinib, momelotinib, fedratinib and ruxolitinib, or a pharmaceutically acceptable salt thereof.

Aspect 20. The method of aspect 17, wherein the JAK inhibitor is ruxolitinib, or a pharmaceutically acceptable salt thereof.

Aspect 21. The method of aspect 17, wherein the GSK-3β inhibitor, or pharmaceutically acceptable salt thereof, is administered to the patient in a range of from about 1 mg/kg to about 50 mg/kg.

Aspect 22. The method of aspect 17, wherein the GSK-3β inhibitor, or pharmaceutically acceptable salt thereof, is administered to the patient in a range of from about 5 mg/kg to about 15 mg/kg.

Aspect 23. The method of aspect 17, wherein about 9 mg/kg of the GSK-3β inhibitor, or pharmaceutically acceptable salt thereof, is administered to the patient.

Aspect 24. The method of aspect 17, wherein the GSK-3β inhibitor, or pharmaceutically acceptable salt thereof, is administered to the patient once per week during a 28-day treatment cycle.

Aspect 25. The method of aspect 17, wherein the GSK-3β inhibitor, or pharmaceutically acceptable salt thereof, is administered to the patient twice per week during a 28-day treatment cycle.

Aspect 26. The method of aspect 17, wherein the GSK-3β inhibitor, or pharmaceutically acceptable salt thereof, is administered to the patient on days 1 and 4 of the week.

Aspect 27. The method of aspect 17, wherein the GSK-3β inhibitor, or pharmaceutically acceptable salt thereof, is administered to the patient intravenously.

Aspect 28. The method of aspect 17, wherein the JAK inhibitor, or pharmaceutically acceptable salt thereof, is administered to the patient in a range of from about 1 mg to about 50 mg.

Aspect 29. The method of aspect 17, wherein the JAK inhibitor, or pharmaceutically acceptable salt thereof, is administered to the patient in amounts of:
about 5 mg twice a day for patients with a platelet count ≥20,000/mL; or
about 10 mg twice day for patients with a platelet count ≥50,000/mL; or
about 15 mg twice a day for patients with platelet count ≥100,000/mL; or
about 20 mg twice a day for patients with platelet count ≥200,000/mL.

Aspect 30. The method of aspect 17, wherein the JAK inhibitor, or pharmaceutically acceptable salt thereof, is administered to the patient twice per day during a 28-day treatment cycle.

Aspect 31. The method of aspect 17, wherein the JAK inhibitor, or pharmaceutically acceptable salt thereof, is administered to the patient orally.

Aspect 32. A method of treating myelofibrosis in a patient, comprising administering to the patient a therapeutically effect amount of:

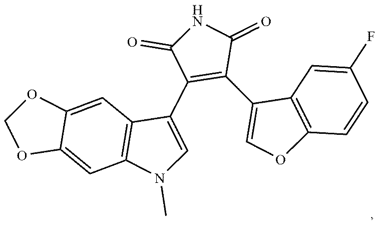

or a pharmaceutically acceptable salt thereof.

Aspect 33. The method of aspect 32, wherein the

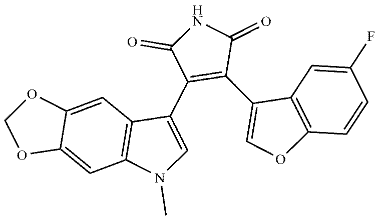 [SMILES: Cn1cc(C2=C(c3coc4ccc(F)cc34)C(=O)NC2=O)c2cc3c(cc21)OCO3]

or a pharmaceutically acceptable salt thereof, is administered , to the patient in a range of from about 5 mg/kg to about 15 mg/kg.

Aspect 34. The method of aspect 32 or aspect 33, wherein about 9 mg/kg of the

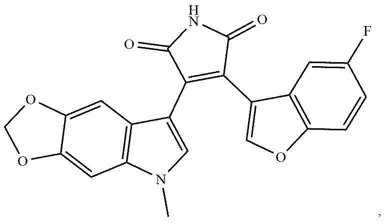

or oharmaceuticallv acceptable salt thereof, is administered to the patient.

Aspect 35. The method of any of aspects 32-34, wherein the

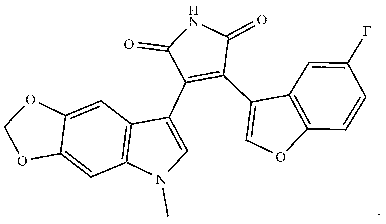

or pharmaceutically acceptable salt thereof, is intravenously administered to the patient on days 1 and 4 of each week during a 28-day treatment cycle.

Aspect 36. The method of any of aspects 32-35, further comprising administering to the patient a therapeutically effect amount of ruxolitinib, or a pharmaceutically acceptable salt thereof.

Aspect 37. The method of aspect 36, wherein the ruxolitinib, or pharmaceutically acceptable salt thereof, is orally administered to the patient in amounts of:
about 5 mg twice a day during a 28-day treatment cycle for patients with a platelet count ≥ 20,000/mL; or
about 10 mg twice day during a 28-day treatment cycle for patients with a platelet count ≥ 50,000/mL; or
about 15 mg twice a day during a 28-day treatment cycle for patients with platelet count ≥ 100,000/mL; or
about 20 mg twice a day during a 28-day treatment cycle for patients with platelet count ≥ 200,000/mL.

Aspect 38. A method of treating myelofibrosis in a patient, comprising administering to the patient a therapeutically effect amount of:

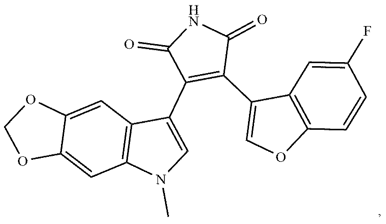

or a pharmaceutically acceptable salt thereof, in combination with a therapeutically effect amount of ruxolitinib, or a pharmaceutically acceptable salt thereof.

Aspect 39. The method of aspect 38, wherein the

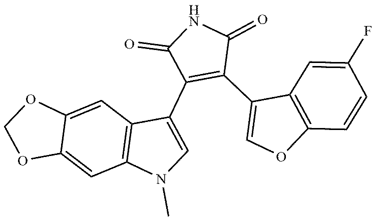 [SMILES: Cn1cc(C2=C(c3coc4ccc(F)cc34)C(=O)NC2=O)c2cc3c(cc21)OCO3]

or pharmaceutically acceptable salt is adminsitered , thereof,to the patient in a range of from about 5 mg/kg to about 15 mg/kg.

Aspect 40. The method of aspect 38 or aspect 39, wherein about 9 mg/kg of the

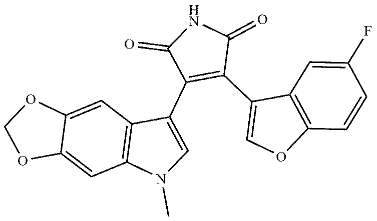 [SMILES: Cn1cc(C2=C(c3coc4ccc(F)cc34)C(=O)NC2=O)c2cc3c(cc21)OCO3]

, or pharmaceutically acceptable salt thereof is administered to the patient.

Aspect 41. The method of any of aspects 38-40, wherein the

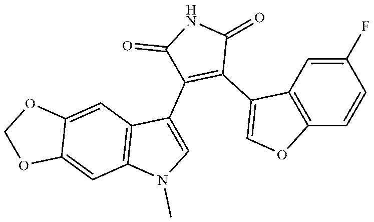 [SMILES: Cn1cc(C2=C(c3coc4ccc(F)cc34)C(=O)NC2=O)c2cc3c(cc21)OCO3]

, or pharmaceutically acceptable salt thereof, is intravenously administered to the patient on days 1 and 4 of each week during a 28-day treatment cycle.

Aspect 42. The method of any of aspects 38-41, wherein the ruxolitinib, or pharmaceutically acceptable salt thereof, is orally administered to the patient in amounts of:
about 5 mg twice a day during a 28-day treatment cycle for patients with a platelet count ≥ 20,000/mL; or
about 10 mg twice day during a 28-day treatment cycle for patients with a platelet count ≥ 50,000/mL; or
about 15 mg twice a day during a 28-day treatment cycle for patients with platelet count ≥ 100,000/mL; or
about 20 mg twice a day during a 28-day treatment cycle for patients with platelet count ≥ 200,000/mL.

Aspect 43. A kit comprising

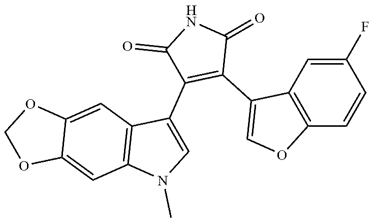 [SMILES: Cn1cc(C2=C(c3coc4ccc(F)cc34)C(=O)NC2=O)c2cc3c(cc21)OCO3]

or a pharmaceutically acceptable salt thereof, and ruxolitinib, , or a pharmaceutically acceptable salt thereof.

Aspect 44. The kit of aspect 43, further comprising a set of instructions for using the kit in a method of treating myelofibrosis.

1. A method of treating myelofibrosis in a patient, comprising administering to the patient a therapeutically effect amount of a glycogen synthase kinase-3 beta (GSK-3β) inhibitor, or a pharmaceutically acceptable salt thereof.

2. The method of claim 1, wherein the GSK-3β inhibitor is: 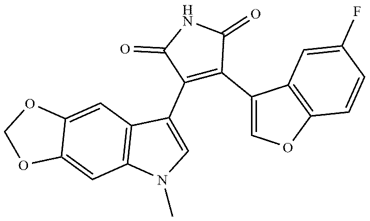 [SMILES: Cn1cc(C2=C(c3coc4ccc(F)cc34)C(=O)NC2=O)c2cc3c(cc21)OCO3]
or a pharmaceutically acceptable salt thereof.

3. The method of claim 1, wherein the GSK-3β inhibitor, or pharmaceutically acceptable salt thereof, is administered to the patient in a range of from about 1 mg/kg to about 50 mg/kg.

4. The method of claim 1, wherein the GSK-3β inhibitor, or pharmaceutically acceptable salt thereof, is administered to the patient in a range of from about 5 mg/kg to about 15 mg/kg.

5. The method of claim 1, wherein about 9 mg/kg of the GSK-3β inhibitor, or pharmaceutically acceptable salt thereof, is administered to the patient.

6. The method of claim 1, wherein the GSK-3β inhibitor, or pharmaceutically acceptable salt thereof, is administered to the patient once per week during a 28-day treatment cycle.

7. The method of claim 1, wherein the GSK-3β inhibitor, or pharmaceutically acceptable salt thereof, is administered to the patient twice per week during a 28-day treatment cycle.

8. The method of claim 1, wherein the GSK-3β inhibitor, or pharmaceutically acceptable salt thereof, is administered to the patient on days 1 and 4 of the week.

9. The method of claim 1, wherein the GSK-3β inhibitor, or pharmaceutically acceptable salt thereof, is administered to the patient intravenously.

10. The method of claim 1, further comprising administering to the patient a therapeutically effect amount of a JAK inhibitor, or a pharmaceutically acceptable salt thereof.

11. The method of claim 10, wherein the JAK inhibitor is selected from the group consisting of pacritinib, momelotinib, fedratinib and ruxolitinib, or a pharmaceutically acceptable salt thereof.

12. The method of claim 10, wherein the JAK inhibitor is ruxolitinib, or a pharmaceutically acceptable salt thereof.

13. The method of claim 10, wherein the JAK inhibitor, or pharmaceutically acceptable salt thereof, is administered to the patient in a range of from about 1 mg to about 50 mg.

14. The method of claim 10, wherein the JAK inhibitor, or pharmaceutically acceptable salt thereof, is administered to the patient in amounts of:
about 5 mg twice a day for patients with a platelet count ≥ 20,000/mL; or
about 10 mg twice day for patients with a platelet count ≥ 50,000/mL; or
about 15 mg twice a day for patients with platelet count ≥ 100,000/mL; or
about 20 mg twice a day for patients with platelet count ≥ 200,000/mL.

15. The method of claim 10, wherein the JAK inhibitor, or pharmaceutically acceptable salt thereof, is administered to the patient twice per day during a 28-day treatment cycle.

16. The method of claim 10, wherein the JAK inhibitor, or pharmaceutically acceptable salt thereof, is administered to the patient orally.

17-37. (canceled)

38. A method of treating myelofibrosis in a patient, comprising administering to the patient a therapeutically effect amount of: 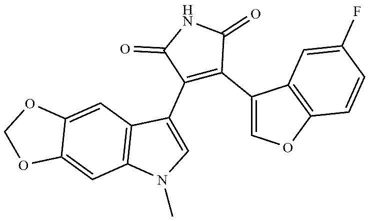 [SMILES: Cn1cc(C2=C(c3coc4ccc(F)cc34)C(=O)NC2=O)c2cc3c(cc21)OCO3]
or a pharmaceutically acceptable salt thereof, in combination with a therapeutically effect amount of ruxolitinib, or a pharmaceutically acceptable salt thereof.

39. The method of claim 38, wherein the 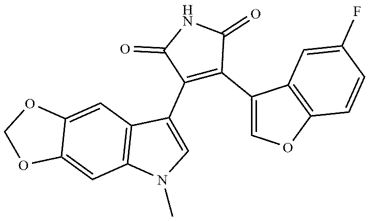 [SMILES: Cn1cc(C2=C(c3coc4ccc(F)cc34)C(=O)NC2=O)c2cc3c(cc21)OCO3]
or pharmaceutically acceptable salt thereof, is administered to the patient in a range of from about 5 mg/kg to about 15 mg/kg.

40. The method of claim 38 , wherein about 9 mg/kg of the 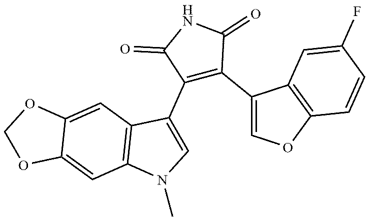 [SMILES: Cn1cc(C2=C(c3coc4ccc(F)cc34)C(=O)NC2=O)c2cc3c(cc21)OCO3]
or pharmaceutically acceptable salt thereof, is administered to the patient.

41. The method of claim 38,wherein the 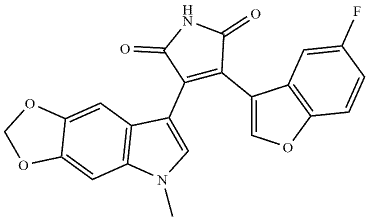 [SMILES: Cn1cc(C2=C(c3coc4ccc(F)cc34)C(=O)NC2=O)c2cc3c(cc21)OCO3]
or pharmaceutically acceptable salt thereof, is intravenously administered to the patient on days 1 and 4 of each week during a 28-day treatment cycle.

42. The method of claim 38,wherein the ruxolitinib, or pharmaceutically acceptable salt thereof, is orally administered to the patient in amounts of:
about 5 mg twice a day during a 28-day treatment cycle for patients with a platelet count ≥ 20,000/mL; or
about 10 mg twice day during a 28-day treatment cycle for patients with a platelet count ≥ 50,000/mL; or
about 15 mg twice a day during a 28-day treatment cycle for patients with platelet count ≥ 100,000/mL; or
about 20 mg twice a day during a 28-day treatment cycle for patients with platelet count ≥ 200,000/mL.

43-44. (canceled)
